(19) United States
(12) Patent Application Publication
(10) Pub. No.: US 2023/0270667 A1
(43) Pub. Date: Aug. 31, 2023

(54) CANNABIDIOL INFUSED CANDLE

(71) Applicants: Durrelle Darren Gardner, Philadelphia, PA (US); Darryl Ethan Gardner, Philadelphia, PA (US)

(72) Inventors: Durrelle Darren Gardner, Philadelphia, PA (US); Darryl Ethan Gardner, Philadelphia, PA (US)

(21) Appl. No.: 17/822,198

(22) Filed: Aug. 25, 2022

Related U.S. Application Data
(60) Provisional application No. 63/314,098, filed on Feb. 25, 2022.

(51) Int. Cl. A61K 9/00; A61K 31/05; A61K 36/258; A61K 47/44; C11C 5/02; C11C 5/00

(52) U.S. Cl. CPC A61K 9/007; A61K 31/05; A61K 36/258; A61K 47/44; C11C 5/023; C11C 5/006

(57) ABSTRACT

A candle including a composition of soy wax, cannabidiol oil, and fragrances, and a wick coated with a cannabidiol oil, positioned to extend partially therefrom the composition.

### CROSS-REFERENCE TO RELATED APPLICATION

This application claims the benefit of priority of U.S. provisional application No. 63/314,098, filed Feb. 25, 2022, the contents of which are herein incorporated by reference.

### BACKGROUND OF THE INVENTION

The present invention relates to candles and, more particularly, to cannabidiol infused candles.

Many people today suffer from anxiety due to stress from work or other circumstances. This anxiety is difficult to counter. Also, aches and pains from injuries, prior surgical procedures, and hereditary illnesses make life extremely challenging for many.

Presently, medical marijuana is inhaled through a pipe, cigarette, or smoking device. These medical marijuana devices require inhaling directly from the device which directly contributes to possible damage to the teeth and throat. Although currently available present devices may offer some relief, the probability of teeth throat damage is high

As can be seen, there is a need for a need for a device that enables a user to experience the benefits of inhaling marijuana without the negative consequences such as teeth or throat damage.

### SUMMARY OF THE INVENTION

In one aspect of the present invention, a candle comprises a composition comprising soy wax, cannabidiol oil, and a fragrance, and a wick coated with a cannabidiol oil, positioned to extend partially therefrom the composition.

These and other features, aspects and advantages of the present invention will become better understood with reference to the following drawings, description, and claims.

### BRIEF DESCRIPTION OF THE DRAWINGS

FIG. 1 is a perspective view of a candle according to an embodiment of the present invention;

FIG. 2 is a sectional view taken on line 2-2 of FIG. 1; and

FIG. 3 is a flow chart showing method steps according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims with reference to the drawings.

A general overview of the various features of the invention will be provided, with a detailed description following. Broadly, an embodiment of the present invention provides a cannabis infused wax candle intended to promote healing while relieving stress and anxiety. In some embodiments of the present invention, a wax candle emits cannabidiols and chemical compounds to be inhaled by the recipient.

By emitting amounts of cannabinoids along with other ingredients (such as rose hips, chamomile, ginseng etc.) into the air when burned, the chemicals in a candle of the present invention may aid a recipient in relaxation, reduce anxiety, and provide relief from aches, pains, and discomfort from injuries.

Direct inhalation of a combination of natural and healthy ingredients may provide relief from aches and pains. The present invention may provide a cleaner and healthier experience for the direct inhalation of said ingredients.

In some embodiments, the present invention comprises soy wax, cannabidiol oil, a treated oil wick, and fragrance. In some embodiments of the present invention, the cannabis may be infused into candle and/or a wick.

Elements such as Asian ginseng may be included to provide health improvements including sexual health improvements. Fragrances, such as chamomile, lavender, lemon, etc. are interchangeable and optional dependent upon the preference of the recipient. Fragrances are not particularly limited by the present invention and any fragrances may be used.

Soy wax may be 100% natural, produces less soot and toxins than other waxes, and burn slower allowing healing properties of the cannabidiol and other natural ingredients to slowly and effectively work in conjunction as they enter a system of the recipient in a comfortable and relaxing process.

When lit, the slow burning soy wax candle slowly emits the chemical compound cannabidiol and the herb chamomile oil/extract or ginseng oil/extract with the desired fragrance into the environment to be gently inhaled by the recipient.

The present invention may comprise cannabidiols (CBD), chamomile, and/or ginseng. Cannabidiol is widely prescribed by health professionals to aid in pain management for patients. Chamomile is a natural herb widely used by many in teas for its calming and relaxing abilities. Ginseng is well documented for its many health benefits. Chamomile is also used in skin products as an antioxidant to reduce signs of aging. When these elements or a combination thereof are distributed to be inhaled, they may produce temporary healing and a relaxing state for the recipient

In forming the present invention, soy wax may be heated to 185 degrees then removed from a heating source. Fragrances (chamomile, lavender etc.) are added along with cannabidiol oil. As the wax cools, Asian ginseng may be added. In some embodiments, a wick is treated with cannabidiol oil and inserted into the wax prior to cooling.

To treat the wick, the wick may be dipped or soaked in CBD oil. The wick may be thoroughly soaked in CBD oil for approximately 30 minutes. The wicks may be dried before installation into the candle. The oil may be heated before placing the wicks in the oil.

In some embodiments, the recipient may first find an open space to place the present invention where he or she can relax and be comfortable. The candle may be placed in a space that is open and safe from items that may be flammable (paper, clothing, curtains etc.) or combustible and out of the reach of children. After the candle is lit, the recipient may sit approximately one to three feet away from the burning candle for approximately one hour to two hours or when the desired level of comfort is achieved from the compounds inhaled from the burning candle. Once the desired effects are achieved the candle may be blown out and left in an open space away from flammable and combustible objects to while it cools.

In some embodiments of the present invention, the composition may comprise 1 part soy wax and 1 part CBD oil. In some embodiments of ginseng and/or chamomile may be combined/mixed with the composition at a ratio of 0.75-1.5 parts of ginseng and/or chamomile to 1 part of soy wax and 1 part CBD oil. A fragrance may be added at a ratio of 0.5 parts fragrance to 1 part soy wax and 1 part CBD oil.

For example, two ounces of soy wax, two ounces of CBD oil, 1.5 ounces of ginseng oil, 1.5 ounces of chamomile oil, and 1 ounce of fragrance may be combined to form the composition of the present invention.

In some embodiments, CBD oil, ginseng oil, chamomile oil, and fragrance may be added to the composition at a temperature of approximate 110 degrees Fahrenheit as the soy wax cools from a heated temperature.

The candle of the present invention may also aid in drug and alcohol rehabilitation as a result of its ability to aid in pain management, anxiety, and relaxation.

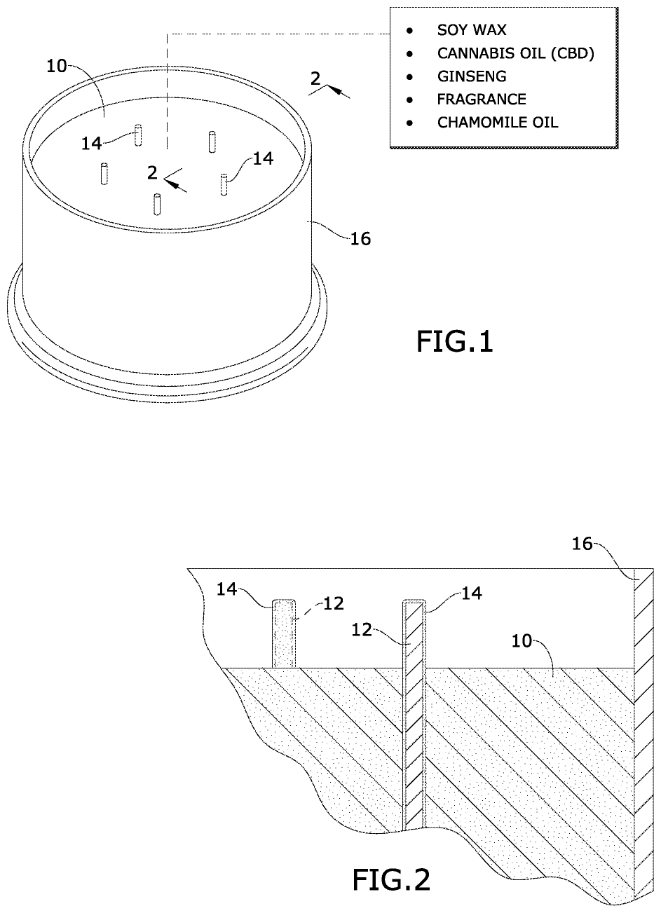

Referring now to the Figures, FIGS. 1 and 2 depict a candle according to an embodiment of the present invention. The candle comprises infused soy wax 10, wicks 12, a cannabidiol (CBD) coating 14 on the wicks 12, and a container 16. As shown in FIG. 1, the wicks 12 extend from a surface of the candle. As the wicks 12 burn, it melts the infused soy wax 10, releasing elements and fragrances infused within the infused soy wax 10.

As best seen in FIG. 2, a CBD coating 14 coats the wicks 12. This CBD coating 14 burns when the wicks 12 are lit. When burned, the CBD coating 14 releases CBD into the air for a recipient to inhale.

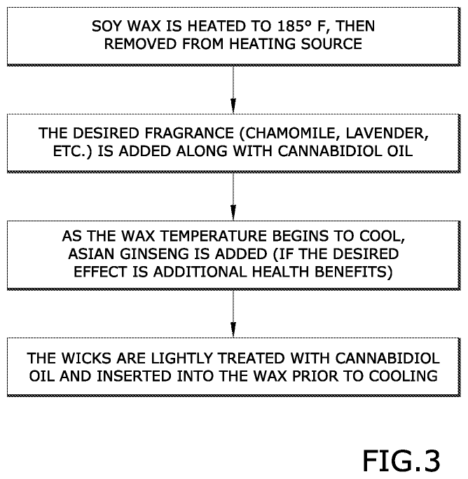

FIG. 3 depicts method steps according to an embodiment of the present invention. Soy wax is infused by heating it to approximately 185 degrees Fahrenheit. Fragrances and CBD oil may then be added while the soy wax is in a heated state forming the infused soy wax 10. Ginseng may be added as the soy wax cools. Wicks 12 may be treated with CBD oil forming the CBD coating 14 on the wicks 12. The wicks 12 may then be inserted into the infused soy wax 10.

It should be understood, of course, that the foregoing relates to exemplary embodiments of the invention and that modifications may be made without departing from the spirit and scope of the invention as set forth in the following claims.

What is claimed is:

1. A candle comprising:
a composition comprising one part soy wax, one part cannabidiol (CBD) oil, and 0.5 parts fragrance; and
a wick coated with a CBD oil, positioned to extend partially therefrom the composition.

2. The candle of claim 1, wherein the composition further comprises Asian ginseng.

3. The candle of claim 1, wherein the composition further comprises chamomile oil.

4. The candle of claim 1, wherein the wick is coated with CBD by a method of soaking the wick in CBD oil for approximately 30 minutes.

5. The candle of claim 1, wherein the wick is coated with CBD by a method of dipping the wick in CBD oil.

6. A method of forming a candle comprising:
heating soy wax to approximately 185 degrees Fahrenheit;
cooling the soy wax to approximately 110 degrees Fahrenheit;
mixing the soy wax with cannabidiol (CBD) oil and fragrances;
coating a wick with CBD oil; and
inserting the wick into the soy wax.

7. The method of claim 6, further comprising mixing Asian ginseng into the soy wax as the soy wax cools.
